(19) United States
(12) Patent Application Publication
(10) Pub. No.: US 2024/0057635 A1
(43) Pub. Date: Feb. 22, 2024

(54) LOW LIPID CONTENT OAT PROTEIN COMPOSITION WITHOUT TRACES OF ORGANIC SOLVENT

(71) Applicant: ROQUETTE FRERES, Lestrem (FR)

(72) Inventors: Leon ZHOU, Geneva, IL (US); Ron CHENG, Geneva, IL (US); Kerry CAMPBELL, Geneva, IL (US)

(21) Appl. No.: 18/259,957

(22) PCT Filed: Jan. 4, 2022
(86) PCT No.: PCT/EP2022/025002

§ 371 Date: Jun. 29, 2023

(30) Foreign Application Priority Data
Jan. 4, 2021 (EP) 21305003.2

(51) Int. Cl. A23J 1/12; A23J 3/14; A23L 33/185; A23K 20/147; A23L 2/66

(52) U.S. Cl. CPC A23J 1/12; A23J 3/14; A23L 33/185; A23K 20/147; A23L 2/66

(57) ABSTRACT

The invention pertains to the field of oat protein compositions and production method thereof. In particular, the present invention also concerns an oat protein composition having low lipid content, which does not contain traces of organic solvent. The present invention also concerns a method of production of an oat protein composition.

### TECHNICAL FIELD

The invention pertains to the field of oat protein compositions and production method thereof. In particular, the present invention also concerns an oat protein composition having low lipid content, which does not contain traces of organic solvent. The present invention also concerns a method of production of an oat protein composition.

### BACKGROUND ART

Oats are a well-known source of a wide variety of useful products. Examples of such products are flour, starch, protein isolate and concentrate, protein-enriched flour, bran, gum and oil. Traditional techniques used in the cereal grain processing industry are frequently difficult to use with oats because of process problems relating to the presence of lipids in the oats. Moreover, unless the oats are de-oiled prior to milling, milling processes would result in the formation of flour and protein fractions containing lipids, which may result in the development of rancidity on storage of the flour and protein.

The most widely used technique consists in a first de-oiling made with help of organic solvents like hexane or ethanol. Man skilled in the art is aware for example of EP0051943 from DUPONT which teaches the use of aliphatic hydrocarbon solvent to remove lipids from oat flours. Main drawbacks of such technologies are industrial use of organic solvent, associated explosion risks and spoilage, and residual levels of lipids in final products.

Such risks seem so important that the main current commercial product called PROATEIN® is currently produced without de-oiling. EP1706001 is only based on the use of amylases and centrifugal separation. As disclosed in the example part, such process leads to a composition where lipids and proteins are not separated, thus leading to a composition having a lipid content much more than 10% by weight based on total weight.

To address these drawbacks, some alternative processes have been recently proposed. Such processes are based on the use of supercritical CO₂. The process of EP2120604 from VALTION TEKNILLINEN describes, as one of the different fractions, a protein composition which is deoiled. This process is done to avoid “complicated wet methods which affect the properties of oat” and is done to maintain the properties of the desired valuable components as natural as possible. However, by this process, the small fraction of protein needs to be processed and grinded, thereby leading to a mean particle size of protein below 10 microns. This process leads to a superfine size protein powder which is not desirable in some applications, but also which is difficult to handle in industrial plants, mainly due to dust formation and explosion hazard. Another major industrial problem linked to particles having a size below 10 microns is that the cyclone and filtration systems needed to recover such small particles are expensive and difficult to operate efficiently and/or effectively. Most importantly supercritical CO₂ is not used at industrial scale of oat components extraction because of difficulties to handle and huge costs due to quantities of supercritical CO₂ that would be needed. Further, this process did not demonstrate its ability to obtain compositions having a high level of protein (e.g. above 70% or above 80%).

The document Brückner-Gühmann et al. (Foaming characteristics of oat protein and modification by partial hydrolysis, European Food Research and Technology, Vol. 244, n° 12, 28 Aug. 2018, pages 2095-2106) describes the production of an oat protein isolate using an oat protein concentrate as a starting material, using a step of alkaline extraction of this concentrate, a step of separation of the protein into the supernatant and a step of lyophilisation of this supernantant to produce the oat protein isolate powder. This article explores the functionality of foaming of the obtained protein isolate. This document does not disclose the mean particle size of the oat protein composition obtained and the composition is not spray dried.

The unpublished PCT application PCT/EP2020/068658 discloses a process for producing an oat protein composition which has a residual lipid content below 10%. The process comprises the adjunction of an amylase enzyme to the protein-rich suspension in order to hydrolyze starch in the protein rich suspension. If the document discloses a spray dried oat protein powder, but does not provide any details about the spray-drying step. Therefore, unpublished PCT application PCT/EP2020/068658 does not disclose the suitability of the protein composition to be spray dried efficiently. When spray drying a composition, its suitability to be spray dried allows high productivity when spray drying or limitation of product lost in the spray drier during the process.

### DESCRIPTION OF THE INVENTION

It is one of the achievements of the invention to provide a powder of an oat protein composition. Another embodiment of the invention concerns the process of manufacturing an oat protein composition with low lipid content that does not need the use of solvent extraction or supercritical CO₂. One additional advantage of this process is that it maintains the oat starch native during the process of extraction.

More precisely, the invention concerns in a first embodiment of the present invention is a powder of an oat protein composition characterized in that said composition does not contain any traces of organic solvent, has protein content higher than 55%, has a extractable lipid content below 10% by weight on dry matter based on the total dry weight of the oat protein composition, a starch:soluble fiber mass ratio above 1 and has a mean particle size d-50 greater than 10 microns.

By “oat protein composition”, it is meant a composition comprising essentially oat protein as the only source of protein. In other terms, the oat protein composition does not comprise any protein that comes from another origin than oat.

By “a composition that does not contain traces of organic solvent”, it is meant a composition that contains less than 100 ppm of solvent, preferably less than 10 ppm of organic solvent and more preferably a composition that does not contain organic solvent at all.

By “organic solvent”, it is meant solvent based on compounds that contain carbon. On the opposite, inorganic solvents which are allowed in this invention do not contain carbon. A typical inorganic solvent allowed in the present invention is water.

“Oat” in the present application must be understood as a cereal plant belonging to the botanical genus Avena. This genus can be divided in wild and cultivated species which have been cultivated for thousands of years as a food source for humans and livestock. The cultivated species contain:
Avena sativa—the most cultivated specie, commonly referred to as “oats”.
Avena abyssinica—the Ethiopian oat, native to Ethiopia, Eritrea, and Djibouti, naturalized in Yemen and in Saudi Arabia
Avena byzantina, a minor crop in Greece and Middle East, introduced in Spain, Algeria, India, New Zealand, South America, etc.
Avena nuda—the naked oat or hulless oat, which plays the same role in Europe as does A. abyssinicain Ethiopia. It is sometimes included in A. sativa and was widely grown in Europe before the latter replaced it. As its nutrient content is somewhat better than that of the common oat, A. nuda has increased in significance in recent years, especially in organic farming.
Avena strigosa—the lopsided oat, bristle oat, or black oat, grown for fodder in parts of Western Europe and Brazil.

In a preferred embodiment, oat protein composition is a protein concentrate, or a protein isolate. The oat protein composition can thus have around 50% by weight of protein or above, based on dry matter based on the total dry weight of the oat protein composition, for example from around 55 to 85%.

In the present application, “protein concentrate” must be understood as an oat protein composition which contains from 50% to 70%, by weight of protein on dry matter based on the total dry weight of the oat protein composition.

In the present application, “protein isolate” must be understood as an oat protein composition which contains more than 70%, generally more than 75%, preferably more than 80% by weight of protein on dry matter based on the total dry weight of the oat protein composition. The protein isolate can comprise less than 95%, generally less than 90% of protein on dry matter based on the total dry weight of the oat protein composition.

Various protocols can be used from prior art in order to quantify the protein content. In the present application, a preferred method to quantify the protein content consists of 1) determining the nitrogen content in the composition and 2) multiplying the nitrogen content by 6,25 factor (which represent the average quantity of nitrogen in protein). The nitrogen content can be determined by any suitable method in the art, such as the Kjeldhal method or by using a combustion analyzer. Preferably, the nitrogen content is determined by a combustion analyzer.

In the present application “protein” must be understood as molecules, consisting of one or more long chains of amino-acid residues. In the present application, proteins can be native proteins or modified proteins, including hydrolyzed proteins. These proteins can be present in different concentrations, including protein isolates or protein concentrates. Oats are the only cereal containing avenalin as globulin or legume-like protein, as the major storage protein (80% by weight). Globulins are generally characterized by their solubility in dilute saline as opposed to the more typical cereal proteins, such as gluten and zein which is a prolamine. The minor protein of oat is the prolamine which is called avenin.

In the present application “extractable lipid” must be understood as molecules that are soluble in nonpolar solvents for example petroleum ether, i.e. extractable lipids. Lipids include fatty acids, waxes, sterols, fat-soluble vitamins (such as vitamins A, D, E, and K), monoglycerides, diglycerides and triglycerides. Oats, after corn, have the highest lipid content of all the cereals, i.e. greater than 6%, sometimes greater than 10% by weight for some oats, in comparison to about 2-3% by weight for wheat and most other cereals.

One advantage of the present invention is even the lipids that are not soluble in non-polar solvents can also be eliminated, leading to a oat protein composition having a the total lipids content is also low.

The total lipid content used for the invention is acid hydrolysis using AOAC 996.06 method, while extractable lipid is measured by Soxhlet method using petroleum ether using AOAC 963.15 protocol.

The oat protein composition of the invention comprises an extractable lipid content below 10% by weight on dry matter based on the total dry weight of the oat protein composition, advantageously below 9%, more advantageously below 8%, even more advantageously below 7%, preferentially below 6%. The oat protein composition of the invention may comprise an extractable lipid content above 1% by weight on dry matter based on the total dry weight of the oat protein composition, for example more than 2%. The oat protein composition of the invention comprises an total lipid content below 10% by weight on dry matter based on the total dry weight of the oat protein composition, advantageously below 9%, more advantageously below 8%. The oat protein composition of the invention may comprise a total lipid content above 1% by weight on dry matter based on the total dry weight of the oat protein composition, for example more than 2%.

The oat protein composition can comprise from 2 to 25% by weight of starch on dry matter based on the total dry weight of the oat protein composition, preferably from 3 to 20%, more preferably from 3 to 12%. Starch content of the composition can be determined using AOAC Official Method 996.11, Starch (Total) in Cereal Products, and more particularly using the method of the booklet Megazyme, Total starch assay procedure (amyloglucosidase/α-amylase method) K-TSTA-50A/K-TSTA-50A 11/20, AOAC 996.11.

The oat protein composition can comprise a soluble fiber content going below 10% by weight on dry matter based on the total dry weight of the oat protein composition, preferably from 0.1 to 5%, more preferably from 0.1 to 3%. In the present application, soluble fiber content, insoluble fiber content, fiber content (which includes the total of soluble and insoluble fiber contents) can be determined using AOAC Official Method 2017.16, Total Dietary Fiber in Foods and Food Ingredients. By soluble fibers, it is meant to be fibers soluble in ethanol as described in this method. One of the dietary fibers generally present in the composition is beta-glucans.

The present composition contains, on a dry substance basis, more starch than soluble fiber, i.e. has a starch:soluble fiber mass ratio above 1.

The oat protein composition can comprise other components than protein, starch, lipid and soluble fiber, the total of these other components being generally in a quantity than lower 10%, more generally lower than 5%, based on the total dry substance of the composition. These other components do comprise for example minerals, starch hydrolysate (starch hydrolysate should be understood as “maltodextrins” and this content can be determined using the booklet Megazyme cited above) or insoluble fiber.

The powder of oat protein composition presents advantageously a mean particle size greater than 20 microns, preferably greater than 30 microns, more preferably greater than 40 microns. The oat protein composition presents advantageously a mean particle size lower than 300 microns, preferably lower than 200 microns, more preferably lower than 150 microns.

In the present application “particle size” must be understood as a notion introduced for comparing dimensions of solid, liquid or gaseous particles. The particle-size distribution (PSD) of a powder, or granular material, or particles dispersed in fluid, is a list of values or a mathematical function that defines the relative amount, typically by mass, of particles present according to size. Several methods can be used for measuring particle size and particle size distribution. Some of them are based on light, or on ultrasound, or electric field, or gravity, or centrifugation. The use of sieves is a common measurement technique. In the present application, the use of laser diffraction method is preferred. As for “mean particle size” (d 50) determined by laser diffraction, this mean particle size is a volume-weighted mean particle size. The man skilled in the art will be able to select a laser diffraction method allowing him to obtain an accurate mean particle size determination. An example of such method is indicated in the examples section.

In the present application, “dry matter” must be understood as the relative percentage by weight of solids based on total weight of the sample. Every well-known method can be used but desiccation method, which consists of estimating quantity of water by heating a known quantity of sample, is preferred.

The powder composition generally comprise, based on the total weight of the composition, a water content lower than 10%, generally between 3 and 7%.

Based on the total weight of proteins in the composition, the composition can comprise:
from 0,5 to 30% of proteins having a molecular weight of 300 kDa and more, advantageously from 5 to 15%,
from 30 to 75% of proteins having a molecular weight of between 50 and 300 kDa, advantageously from 45 to 65%,
from 10 to 50% of proteins having a molecular weight of between 10 and 50 kDa, advantageously from 25 to 45%,
from 0,5 to 20% of proteins having a molecular weight of 10 kDa and less, advantageously from 1 to 10%,
   the sum making 100%.

An advantage of this preferred embodiment of the invention is that the molecular weight of the oat protein composition is high, which can provide different protein functionalities compared to low molecular weight oat protein composition such as described e.g. in the document Brückner-Gühmann et al. These functionalities can depend on the process of manufacturing that is detailed hereafter.

The protein molecular weight (MW) distribution can be determined using Size Exclusion Chromatography.

To do so, it is possible to use the following method which is indicated as an example. Samples can be dissolved in 200 mM phosphate buffer, pH=7.6, vortexed for 1 minute initially and 10 minutes later and stored at 4 C over-night. The solutions are centrifuged at 7000 g for 10 minutes, the supernatant is measured for soluble protein content the next day, and the samples are diluted to 10 mg/mL with phosphate buffer. The samples are chromatographed using 2 SEC columns (400 and 300 Agilent Advanced Bio SEC Column, 5000-1,250,000 MW Range) in sequence using phosphate buffer, pH=7.6 as the mobile phase at 0.5 mL/minute. The detection is a UV=280 nm. Several protein molecular weight standards going from 14300 to 669000 Da (Lysozyme, Carbonic Anhydrase, BSA, HSA, B-Amylase, Apoferritin, Thyroglobulin) are analyzed to identify the retention time and calibrate the chromatography apparatus. For sample analysis, chromatograms peak or peak apex (group) is determined along with the range of the peak (start and end) and the molecular weight is determined for the range and peak apex. The percent of molecular weight can be determined, for example, for: >300 kDa, 300 kDa to 50 kDa, 50 KDa to 10 KDa and <10 kDa.

In another embodiment, the oat protein composition can be hydrolysed. Hydrolysis can be conducted by any means known, for example by using a protease or a peptidase enzyme.

According to an embodiment of the invention, the oat protein composition of the invention presents an improved color. The powder can have parameters a* between −1 and 1, and L* above 80 when measured in the CIELAB color space. Preferably, the parameter b* is lower than 15, by example lower than 10. L*, a* and b* parameters are known in the art and can be determined using classic color CIELAB color space, for example it can be characterized using a CR-5 device from Konica Minolta following the instructions manual.

A second embodiment of the present invention is a process for producing an oat protein composition which has an extractable lipid content below 10% by weight on dry matter based on the total dry weight of the oat protein composition, which can be the oat protein composition as defined above, characterized in that the process comprises the following steps:
a. preparing a protein-rich suspension from oat starting material;
b. separating a soluble fraction comprising protein from an insoluble fraction comprising starch and fibers;
c. A step of adding a starch hydrolysate and a polysorbate to the soluble fraction and optionally adjusting pH from 5.8 to 8.0, preferably 6-7 to form additive-containing soluble fraction;
d. A step of heating the additive-containing soluble fraction at a temperature going from 35 to 80° C.;
e. A step e) of forming a proteic precipitate;
f. A step of separation of the proteic precipitate from solubles components to obtain a protein curd
g. Optionally at least one washing step of the protein curd, preferably at a pH between 4.5 suggest to reduce to 4.5 for wide range coverage and 6 and at a temperature between 50 and 60° C.
h. Optionally a step of adjusting the pH of the protein curd at a range going from 6.5 to 10.
i. Optionally a step of heat treatment of the protein curd
j. Optionally a step of homogenization treatment
k. Optionally a step of drying including spray drying.

The first step consists in preparing a protein-rich suspension from oat starting material. The oat starting material may comprise oat flour, low-fiber oat flour, oat bran or oat pulp fraction from oat milk production or oat syrup production. The oat starting material typically comprises protein, lipid, fiber and starch. The oat starting material does typically not comprise organic solvents because it is not preliminary treated with these.

Typically, the oat starting material comprises between 5 and 45% protein, between 5 and 80% starch, between 5 and 50% fiber and 3 to 15% extractable lipids, the amounts being expressed as weight % of each component based on the total dry solids of each material, the total amounting to 100%.

Typically, whole oat flour comprises 8-30% protein, 40-80% starch and 5-15% fiber and 3 to 15% extractable lipids.

Typically, low-fiber oat flour (or de-hulled oat flour) comprises 10-30% protein, 45-80% starch, 0 to 4% of fiber and 3-15% extractable lipids.

Typically, oat bran comprises 10-30% protein, 30-70% starch and 10-20% fiber and 4 to 15% extractable lipids.

Typically oat processed material (the oat pulp fraction which is a by-product from oat milk or oat syrup production) comprises 10-45% protein, 5-30% starch and starch hydrolysate and 10-50% fiber and 3 to 15% extractable lipids.

The protein-rich suspension is a suspension of oat flour in water. As water, any food compatible water can be used, but tap water, reverse osmosis water and deionized water are preferred. The aim of step 1 is to reach a dry matter comprised between 5% and 20%, preferably between 10% and 15%, most preferably between 10% and 13% by weight with respect to the total weight of the suspension.

Depending on the oat starting material, this protein-rich suspension can be obtained in different ways.

a) When oat seeds are used, said oat seeds may be cultivated and/or commercially available. Oat seeds may then prepared including possible steps of sieving or dehulling.

Oats seeds may be dry- or wet-heated prior to use. The purpose of dry- or wet-heat is to destroy enzymes including beta-glucanase, lipase and lipoxygenase. Indeed, inactivation of lipase and lipoxygenase is indicated to prevent the product from turning rancid. In the process of the present invention, heat treatment, in particular steaming, should be avoided or at least be kept as short as possible and/or carried out at a temperature as low as possible to keep oat protein denaturation low.

The oat seeds are then ground in order to obtain protein rich flour. To grind oat seeds, all well-known common technologies can be used including stone-mill, roller mill or knife-mill. In this step, preferred particle size distribution of the resulting protein rich flour may be a d50 (50th percentile) above 30 microns, preferably above 40 microns, even more preferably above 50 microns. In the present invention, d50 is measured with help of any known by man skilled in the art technology. In a preferred way, laser granulometry is preferred.

The protein rich flour can comprise a protein content above 14%, e.g. above 16%, based on the dry solids content of the protein flour.

b) It is possible to use directly commercial oat flour. When oat flour is used, typically, the flour may be weighed and introduced in a tank containing water and equipped with agitation, pH and heating apparatus at the desired dry matter content.

c) It is also possible to prepare the suspension from oat bran. Oat bran is made up of only the outer shells of the seed. When oat seed is processed to remove the inedible exterior body of the seed, this leaves behind the oat groat, and oat bran is the outer layer of this oat groat kernel, which is right underneath the inedible grain portion.

d) it is also possible to prepare the suspension of step 1 from oat pulp. Oat pulp, also known as oat okara, is the pulp that is generally discarded during the preparation of oat milk or oat syrup. Briefly, the procedure starts by measuring and milling the oat grains to break apart their outer hull. Then the grains are stirred in warm water and ground into a slurry. The slurry is treated, eventually with enzymes, and heat to create a thick liquid oat base, which is then separated by decanting, filtering, or centrifugation. The supernatant represents the oat milk or syrup, whereas the pellet is the oat pulp or okara.

Preferably, the protein-rich suspension has, at least during a part of the step b), a pH going from 1.5 to 3.0 or from the range 7.0 to 11.0, preferably 2.0-2.5 or 8.5-10.5, even more preferable 8.5-10.5. At alkaline pH, the protein recovery is even higher than in the case of acid extraction. The solvent is preferably water. However, it can be added compounds to make higher the solubility of the protein fraction in the protein-rich suspension. Any inorganic or organic acid and base reactant can be used. They may be chosen from caustic soda, potash, lime, citric acid, ascorbic acid, nitric acid, sulfuric acid and hydrochloric acid. The pH of the protein-rich suspension can be set with these acid and base components to a pH going from 1.5 to 3.0 or from the range 7.0 to 11.0, preferably 2.0-2.5 or 8.5-10.5. The temperature during that steps a and b can be adjusted between 2 and 30° C., preferably between 10° C. and 30° C.

The step of separation b) can be done using classic separation methods, including decantation, filtration or centrifugation techniques. The device to conduct the separation step b) can be a decanter, a disc centrifuge (e.g. Alfa Laval Merco centrifuge), a tubular centrifuge, a basket centrifuge or a rotary vacuum filter. Preferably, a decanter is used for this separation step. At the end of this step is obtained a soluble fraction comprising protein separated from an insoluble fraction comprising starch and fibers. This fraction comprising starch and fibers can be further processed in order to obtain a purified oat starch and a purified oat fiber. Classic separation techniques can be used to obtain these. One advantage of the process of the invention is that it allows to obtain an oat starch product in which the starch is native and non hydrolyzed.

During the step c), a starch hydrolysate and a polysorbate are added, sequentially or simultaneously, to the soluble fraction comprising protein to form a additive-containing soluble fraction. Preferably, the total mass content of starch hydrolysate and polysorbate added during step c), based on the total mass of dry matter of the soluble fraction, is between 0.5 and 10%, preferably between 2 and 4%. The starch hydrolysate:polysorbate ratio can be added in a mass ratio going from 0,66 to 15, by example, from 1 to 10, preferably from 2 to 8, more preferably from 2 to 5.

These starch hydrolysates are also known as maltodextrins or glucose syrups. Preferably, the starch hydrolysate has a low dextrose equivalent (DE), for example lower than 35. The starch hydrolysate can have a dextrose equivalent going from 12 to 30, preferably from 15 to 20. The starch hydrolysate may be obtained by hydrolysis of starch from any source, for example from pea, oat, wheat or corn. It may be a starch hydrolysate commercialized by the applicant under the brand name GLUCIDEX®.

Polysorbates are a class of emulsifiers used in cosmetic, pharmaceuticals and food preparations. Polysorbates are oily liquids derived from ethoxylated sorbitan (a derivative of sorbitol) esterified with fatty acids. Common brand names for polysorbates include Scattics, Alkest, Canarcel, and Tween. Common used polysorbate are Polysorbate 20 (polyoxyethylene (20) sorbitan monolaurate), Polysorbate 40 (polyoxyethylene (20) sorbitan monopalmitate), Polysorbate 60 (polyoxyethylene (20) sorbitan monostearate) and Polysorbate 80 (polyoxyethylene (20) sorbitan monooleate) (number following ‘polyoxyethylene’ refers to total number of oxyethylene —(CH2CH2O)— groups found in the molecule and number following ‘polysorbate’ is related to the type of fatty acid associated with the polyoxyethylene sorbitan part of the molecule). Preferably, the polysorbate is Polysorbate 80 (polyoxyethylene (20) sorbitan monooleate) also known as Tween 80.

Optionally, the pH is adjusted from 5.8 to 8.0, preferably 6.0 to 7.0. Any acid or base already cited above can be used to do so.

The process comprises a step d) of heating the additive-containing soluble fraction at a temperature going from 35 to 80° C., preferably from 60 to 70° C. At these preferred ranges, the separation of oil and protein is made easier. This step of heating can be done under continuous or uncontinuous mixing of the additive-containing soluble fraction. The step of heating the protein suspension may be done during from 30 to 150 minutes, advantageously from 30 to 120 minutes and preferably from 60 to 90 minutes.

The process also comprises a step e) of forming a proteic precipitate. This step may be done by adjusting the pH of the heated additive-containing soluble fraction close to isoelectric point, for example in the range going from 4.5 to 5.8 to form a proteic precipitate, preferably from 5.0 to 5.5. This step can be done at a temperature going from 20 to 80° C., preferably at a temperature going from 50 to 60° C.

After forming the proteic precipitate, the process comprises a step of separation f) of the proteic precipitate from solubles components, preferably using centrifuge, plate frame separate or membrane, to obtain a protein curd.

The process can optionally comprise at least one washing step of the protein curd, by example by resuspending the protein curd in acidic water and by separating again and obtain a washed protein curd. To conduct this washing step, same conditions than the steps e and f described above can be used.

The process can also comprise a homogeneization step of the protein. Homogenization techniques in the field of proteins are known in the art. In particular, the homogenization may be carried out at high pressure. For example, it can be at a pressure of between 2 MPa and 800 MPa, for example between 2 MPa and 250 MPa.

The process can also comprise an ultrasonication step of the protein curd.

The process can also comprise at least one step of adjusting the pH of the protein curd. The protein curd can have its pH adjusted at a range going from 7.0 to 11.0, by example from 9.0 to 10.0.

The process can also comprise a step of heat treatment of the protein curd. The temperature can be from 75 to 180° C. Depending on the temperature, the time of the treatment can be from 0.1 s to 20 minutes, advantageously from 0.1 to 30 s, most preferably between 10 and 20 s.

The process can comprise also a drying step in order to manufacture the oat protein powder of the invention. The drying step can be done by using drum drying or spray drying, including multi-stage spray drying, preferably spray drying. One advantage of the invention is that the composition allows obtaining an excellent productivity when spray drying and/or limitation of product lost in the spray drier during the process. Without being bound to any theory, it may be explained by the particular composition that has lower viscosity than compositions generally spray dried, because of low content of soluble fiber compared to starch content.

The process according to the invention may also comprise a step of modifying the proteins in the oat protein composition by subjecting the composition to enzymatic treatment with a protease or a glutaminase. This step may be carried out preferably at any stage after recovery of the protein curd.

A third and last embodiment of the present invention is the use of the oat protein composition of the present invention or obtained by the process of the present invention, preferably in food, feed, pharmaceutical and cosmetic fields.

Such oat protein composition is particularly suitable for ready to drink beverages, or baking or any other food application such as protein bars, non-dairy beverages, powder mixes, yogurts, cheeses, or meat-like products. Its low lipid content allows an improved organoleptic experience when formulated, Indeed, when a product comprises high amounts of lipids, these undesirable lipids can get oxidized and develop a rancid taste, which negatively affects the organoleptic quality of the product.

Such oat protein composition is particularly suitable for ready to drink beverages or baking or any other food application such as protein bars, non-dairy beverages, powder mixes, yogurts, cheeses, or meat-like products. Its low lipid content allows an improved organoleptic experience when formulated, Indeed, when a product comprises high amounts of lipids, these undesirable lipids can get oxidized and develop a rancid taste, which negatively affects the organoleptic quality of the product.

In general terms, the oat protein composition of the invention can be used in food and beverage products that may include the oat protein composition in an amount of up to 100% by weight relative to the total dry weight of the food or beverage product, for example in an amount of from around 1% by weight to around 80% by weight relative to the total dry weight of the food or beverage product. All intermediate amounts (i.e. 2%, 3%, 4% . . . 77%, 78%, 79% by weight relative to the total weight of the food or beverage product) are contemplated, as are all intermediate ranges based on these amounts.

Beverages include acid beverages, carbonated beverages (including, but not limited to, soft carbonated beverages); non-carbonated beverages (including, but not limited to, soft non-carbonated beverages such as flavored waters, fruit juice and sweet tea or coffee based beverages); beverage concentrates (including, but not limited to, liquid concentrates and syrups as well as non-liquid ‘concentrates’, such as freeze-dried and/or powder preparations). The protein content in the beverage can be very different and the beverage can be a high protein drink. The content is for example between 1 and 12% of the total mass of the beverage, for example between 2 and 10%. Beverages also include milk-like beverages, that can be «barista» type or «coffee creamer» type.

Food products which may be contemplated in the context of the present invention include baked goods; sweet bakery products (including, but not limited to, rolls, cakes, pies, pastries, and cookies); pre-made sweet bakery mixes for preparing sweet bakery products; pie fillings and other sweet fillings (including, but not limited to, fruit pie fillings and nut pie fillings such as pecan pie filling, as well as fillings for cookies, cakes, pastries, waffles, pancakes, muffins and biscuits, confectionary products and the like, such as fat-based cream fillings); desserts such as flan, custard, gelatins and puddings; frozen desserts (including, but not limited to, frozen dairy desserts such as ice cream—including regular ice cream, soft serve ice cream and all other types of ice cream—and frozen non-dairy desserts such as non-dairy ice cream, sorbet and the like); snack bars (including, but not limited to, cereal, nut, seed and/or fruit bars); bread products (including, but not limited to, leavened and unleavened breads, yeasted and unyeasted breads such as soda breads, breads comprising any type of wheat flour, breads comprising any type of non-wheat flour (such as oat, potato, rice and rye flours), gluten-free breads); pre-made bread mixes for preparing bread products; sauces, syrups and dressings; sweet spreads (including, but not limited to, jellies, jams, butters, nut spreads, dulce de leche and other spreadable preserves, conserves and the like); confectionary products (including, but not limited to, jelly candies, soft candies, hard candies, chocolates, caramels and gums); sweetened and un sweetened breakfast cereals (including, but not limited to extruded breakfast cereals, flaked breakfast cereals and puffed breakfast cereals); and cereal coating compositions for use in preparing sweetened breakfast cereals. Other types of food and beverage product may also be contemplated in the context of the present invention. In particular, animal foods (such as pet foods) are explicitly contemplated.

Oat protein can be used in combination with flavours or masking agents.

Oat protein can also be used, eventually after texturization, in meat-like products such as emulsified sausages or plant-based burgers, fish-like products or seafood-like products. It can also be used for making egg substitutes or for the manufacturing of protein containing products such as tofu or tempeh. «Texturized proteins» generally means proteins texturized by extrusion, i.e. especially by dry extrusion to make Textured Vegetable Protein, wet extrusion or high moisture extrusion. Extruders can be single screw extruders, twin screw extruders, multiple screw extruders. Example of multiple screw extruders are planetary extruder or ring-extruder. Other technologies such as shear cell technology, microextrusion or 3D printing can also be used.

The food or beverage product can be used in specialized nutrition, for specific populations, for example for baby or infants, teenagers, adults, elderly people, athletes, people suffering from a disease. It can be meal substitutes formulations, complete nutrition beverages, for example for weight management or in clinical nutrition (for example tube feeding or enteral nutrition).

The oat protein composition can be used as the sole source of protein but also can be used in combination with other plant or animal proteins. These other proteins can be hydrolyzed or not. Generally, these are in the form of isolates or concentrates. The term “plant protein” denotes all the proteins derived from cereals, oleaginous plants, leguminous plants and tuberous plants, and also all the proteins derived from algae and microalgae or fungi, used alone or as a mixture, chosen from the same family or from different families. In the present application, the term “cereals” is intended to mean cultivated plants of the grass family producing edible grains, for instance wheat, rye, barley, maize, sorghum or rice. The cereals are often milled in the form of flour, but are also provided in the form of grains and sometimes in whole-plant form (fodders). In the present application, the term “tubers” is intended to mean all the storage organs, which are generally underground, which ensure the survival of the plants during the winter season and often their multiplication via the vegetative process. These organs are bulbous owing to the accumulation of storage substances. The organs transformed into tubers can be the root e.g. carrot, parsnip, cassava, konjac), the rhizome (e.g. potato, Jerusalem artichoke, Japanese artichoke, sweet potato), the base of the stalk (more specifically the hypocotyl, e.g. kohlrabi, celeriac), the root and hypocotyl combination (e.g. beetroot, radish). For the purposes of the present invention, the term “leguminous plants” is intended to mean any plants belonging to the family Cesalpiniaceae, the family Mimosaceae or the family Papilionaceae, and in particular any plants belonging to the family Papilionaceae, for instance pea, bean, soy, broad bean, horse bean, lentil, alfalfa, clover or lupin. This definition includes in particular all the plants described in any of the tables contained in the article by R. Hoover et al., 1991 (Hoover R. (1991) “Composition, structure, functionality and chemical modification of legume starches: a review” Can. J. Physiol. Pharmacol., 69, pp. 79-92). Oleaginous plants are generally seed-producing plants from which oil is extracted. Oilseed plants can be selected from sunflower, rapeseed, peanut, sesame, pumpkin or flax. The animal protein can be for example egg or milk proteins, such as whey proteins, casein proteins or caseinate. The oat protein composition can thus be used in combination with one or more of these proteins or amino acids in order to improve the nutritional properties of the final product, for example to improve the PDCAAS of the protein or to bring other or modify functionalities.

The oat protein can also be used for the manufacturing of pharmaceutical products or in fermentation, for example for the production of fungi metabolites or cell culture metabolites.

The oat protein composition of the invention can also be used for acidic food products such as yogurts (including, but not limited to, full fat, reduced fat and fat-free dairy yogurts, as well non-dairy and lactose-free yogurts and frozen equivalents of all of these), cheeses or acidic sauces. Acidic food products can have a pH of 3 to 6 when diluted at a dry matter of 10%. The oat protein composition can be used to form of a milk and fermented and/or acidified to provide yogurts and cheeses. These milks can present a dry matter going from 5 to 30%. These milks can comprise other components such as sugars and fats and optional, Yogurts can include stirred yogurts, set yogurts or yogurts to drink. These can be flavoured or not and can include other components such as fruit preparations and/or sweeteners. Cheeses can be process cheese, swiss cheese, string cheese, ricotta, provolone, parmesan, muenster, mozzarella, jack, manchego, blue, fontina, feta, edam, double Gloucester, cheddar, asiago and Havarti. Acidic sauces are for example mayonnaise or ketchup. The invention will be better understood with the following non-exhaustive examples.

### Examples

### Example 1: Oat Protein Isolate Starting from Oat Flour

In this Example, the scale of pilot continuous process is medium-scale. The following protocol was followed: weigh 100 kg Grain Miller #70 oat flour, fill with 1000 L water (Tank size 2000 L) at 37° C. and mix flour gradually into water, make sure all flour dispersed well in water. Adjust the pH of the protein rich suspension to 2.0-2.1 with 2 N HCl while agitating and keep the temperature at 37° C., mix at 500 rpm for 90 minutes. Feed through Decanter (Centrisys) at 12 L/min, 3000 g-force, 3 rpm differential. The pellet (Heavy phase HP1) and supernatant (Light phase LP1) were collected. Take the LP1 fraction, heat the liquor up to 65° C., then add 3% (w/w) of maltodextrin (Glucidex®19) and add 0.8% (w/w) of polysorbate Tween 80, these amounts being based on the dry substance of the LP1 fraction. Adjust to pH 6.5 with 1 N NaOH, keep 66-68° C. and mix at 300 rpm for 60 min. Adjust pH to 5.2 and keep mix at 200 rpm for 15 min to form a protein precipitate. Feed through a disc-centrifuge at 7 L/min, 9800 rpm, discharge the solids every 90 sec. Collect protein curd (PPT1) and Overflow (O/F1). Dilute the protein curd (˜75 kg, target DS 12-14%) into 400 L of hot water (50° C.), mix at 200 rpm for 30 min. Adjust the pH to 5.2 and feed through a disc-centrifuge at 7 L/min, 9800 rpm, discharge the solids every 97 sec. Collect washed protein curd (PPT2) and Overflow (O/F2). Reconstitute the protein curd with water (10% DS), adjust pH to 7, then apply to a Jet cooker (130° C., 20-30 sec holding time). Submit to Spray drying with 180° C. inlet/90° C. outlet, at 12 L/hr feeding rate. Collect spray dried Oat Protein Isolate (OPI).

### Example 2: Oat Protein Isolate Starting from Low Fiber Oat Flour

In this Example, the scale of pilot continuous process is small-scale. The following process was used: Weigh 25 kg low fiber oat flour commercialized by Richardson. Fill with 250 L water, 37° C. in a 380 L jacketed tank and mix flour gradually into water. Adjust the pH to 9.5 with 2 N NaOH while agitating. Keep the temperature at 50° C., 300 rpm for 30 minutes. Feed to Lemitec decanter at 750 mL/min, 3000 g-force, 100 rpm differential. The pellet (Heavy phase HP1) and supernatant Light phase (LP1) fractions were collected. Put the light phase LP1 fraction in a tank and heat up to 65° C., add 4% (w/w) Glucidex 19 maltodextrin and 1% (w/w) Tween 80. Adjust to pH 6.5 with 2 N HCl, keep 65° C. mix at 300 rpm for 60 min. Adjust pH to 5.2, continuously mix at 160 rpm for 15 min to form a protein precipitate. Feed to Clara 20 Disc centrifuge 2-2.5 L/min, 9000 g, with calculated discharge interval time. Collect protein curd (PPT1) and Overflow (O/F1). Dilute the protein curd 21.6 kg (10-12% DS) with 200 L of (target on 1-2% DS solids) hot water (50° C.), adjust pH to 5.2 and mix at 160 rpm for 30 min. Feed to Clara 20 Disc centrifuge 2-2.5 L/min, 9000 g and collect washed protein curd (PPT2) and Overflow (O/F2). Adjust the dry substance of the washed protein curd PPT2 with water to around 11% DS, adjust pH to 9.5 with NaOH and apply to UHT treatment at 154° C., 30 seconds and discharge at 71° C.; Homogenize with 2-stage homogenizer with 1^{st} stage at 400 bar and 2^{nd} stage at 40 bar. Adjust pH to 7 before applying to spray drying with GEA Minor® spray dryer at 220° C. inlet, 90° C. outlet with ˜3 L/hour feeding rate.

### Example 3: Prior Art Process Involving Starch Hydrolysis Using Oat Flour

Weigh 2.5 kg N°70 oat flour from Grain Millers. Fill a feed tank with 25 L water at 40-50° C. Mix flour into water. Adjust pH to 5.4 to 5.5 with 1 N HCl while agitating for 10 min. Add 25 g Liquozyme supra (from Novozyme). Heat on hot plate 70° C., 300 rpm, for 2 hours. Reduce pH to 5.0 with 1 N HCl and stir for 30 min. Feed through a Lemitec centrifuge at 500 mL/min, 580 G, 10 rpm differentia. Add 12.5 L 50° C. water to the curd. Adjust to pH 5.0 with 1 N HCl. Feed through Lemitec 500 mL/min, 3600 rpm, 10 rpm differential. Adjust pH to 7 before applying to spray drying with GEA Minor® spray dryer at 220° C. inlet, 90° C. outlet with ˜3 L/hour feeding rate.

### Example 4: Control Process that does not Use Maltodextrin and Polysorbate

The following process was used: Weigh 18 kg starting material. Fill Lemitec feed tank with 180 L water, 50° C. in a 190 L jacketed tank and mix flour gradually into water. Adjust pH to 9.5 with 3 N NaOH while agitating and keep the temperature at 50° C., 300 rpm for 60 minutes, then start feeding to Lemitec decanter. Feed through Lemitec (700 mL/min, 3000 g-force, 120 rpm differential) and collect pellet and supernatant, denoted Heavy phase (HP1) and Light phase (LP1) fractions. Take LP1 fraction, adjust pH to 5.2, and continuously mix at 160 rpm for 15 min. Feed to Clara 20 Disc centrifuge (2 L/min, 9000 g), collect protein pellet (PPT1) and Overflow (O/F1). Reconstitute the PPT1 with 150 L hot water (45° C.), adjust pH to 5.2 and mix at 160 rpm for 30 min. Feed to Clara 20 Disc centrifuge (2 L/min, 9000 *g), collect protein pellet (PPT2) and Overflow (O/F2). Adjust PPT2 DS to 10% and pH to 9.5, then apply to UHT treatment at 154° C. using direct steam injection, 30 seconds and flash cooling at 71° C., adjust pH to 7 and pass homogenization with 2-stage homogenizer with 1st stage at 400 bar and 2nd stage at 40 bar, feed to spray drying at 220° C. inlet, 90° C. outlet with ˜3 L/hour feeding rate.

### Example 5: Determination of the Properties of Oat Protein Isolates

Methods

% protein: Protein content is % N6.25 and nitrogen content is determined using combustion analyzer-Elementer, with AOAC 997.09 method.

% lipids: extractable lipid content is determined using Soxhlet extraction in petroleum ether using AOAC 963.15 method and total lipid content is determined using AOAC 996.06 method.

% starch: AOAC Official Method 996.11.

% soluble fiber: AOAC official method 2017.16.

% insoluble fiber: AOAC official method 2017.16.

Color L*a*b*: Determined using a device CR-5 from Konica Minolta following the instructions manual.

% Moisture: Weigh 2-3 grams of sample into pre-weighed aluminum drying pan. Place in 130° C. oven for 2 hours, cooled to room temperature, weigh samples again. Moisture determined based on mass loss.

Protein recovery: mass of protein in the isolate obtained before spray drying/mass of protein in the flour×100

d 50 (μm): d 50 is measured by a laser granulometry apparatus (Mastersizer 3000, from Malvern), which measures intensity of scattered light across a range of scattering angles using forward scattering measurement, on a dry powder without dispersion buffer, and using the software of the apparatus with the Mie scattering model to fit the distribution to the measured scattering pattern.

Spray drying yield: mass of dry powder obtained/mass of dry composition injected×100

Average molecular weight and distribution of Mw: determined by Size Exclusion Chromatography

Results

The composition characteristics of the powder of oat protein isolate obtained under each condition are summarized in Table 1 below:

| | Example 1 | Example 2 | Example 3 | Example 4 |
|---|---|---|---|---|
| Protein % dsb | 83.2% | 87.5% | 54.4% | 56% |
| Lipid extractable | 5.4% | 6.7% | 15.9% | 37% |
| % dsb | | | | |
| Lipid total % dsb | 7.1% | 7.9% | | |
| Starch % dsb | 11.1% | 3.1% | | |
| Soluble fiber % dsb | 2.8% | 0.6% | | |
| Insoluble fiber % dsb | 2.7% | 0.6% | | |
| L* | 82.6 | 83.1 | | |
| a* | 0.48 | 0.31 | | |
| b* | 14.3 | 9.5 | | |
| moisture (%) | 4% | 3% | | |
| Protein recovery | 66% | 68% | | |
| d 50 (μm) | 20.7 | 22.3 | | |
| Spray drying yield | 90% | 69% | | |
| Average Mw (Da) | 42780 | 25752 | | |

As can be seen in Table 1, Examples 1 and 2 gave satisfactory results in terms of protein and lipid percentages, whereas prior art processes do not allow to obtain good quality oat protein isolates. The compositions present a lower content of soluble fiber than starch. The color of the powder is excellent compared to the oat proteins compositions on the market.

Regarding Example 2, surprisingly the protein recovery is even a little higher of the one obtained from Example 1 whereas the scale is smaller in the case of Example 2. We can see that in both cases, it would be possible to achieve a protein recovery percentage higher than 70% when scaling-up the process at industrial scale. Regarding the spray drying yield, these yields are very important compared to the size of spray drying equipments and volume of the oat protein composition spray dried.

The process of example 4 does not comprise any step of treatment using starch hydrolysate and polysorbate, otherwise it is a process similar to the ones of example 1 and 2. Without this step, the amount of lipids increases dramatically. This product does not give satisfactory results to be sold as oat protein concentrate.

1) A process for producing an oat protein composition which has an extractable lipid content below 10% by weight on dry matter based on the total dry weight of the oat protein composition wherein the process comprises the following steps:
a. preparing a protein-rich suspension from oat starting material;
b. separating a soluble fraction comprising protein from an insoluble fraction comprising starch and fibers;
c. a step of adding a starch hydrolysate and a polysorbate to the soluble fraction and optionally adjusting pH from 5.8 to 8.0, preferably 6-7 to form additive-containing soluble fraction;
d. a step of heating the additive-containing soluble fraction at a temperature going from 35 to 80° C.;
e. a step of forming a proteic precipitate;
f. a step of separation of the proteic precipitate from soluble components to obtain a protein curd;
g. optionally at least one washing step of the protein curd, preferably at a pH between 4.5 and 6 and at a temperature between 50 and 60° C.;
h. optionally a step of adjusting the pH of the protein curd at a range going from 6.5 to 10;
i. optionally a step of heat treatment of the protein curd;
j. optionally a step of homogenization treatment; and,
k. optionally a step of drying including spray drying.

2) The process of claim 1 wherein the oat starting material comprises oat flour, oat bran or oat pulp fraction from oat milk production or oat syrup production, preferably oat flour.

3) The process of claim 1 wherein the protein-rich suspension has, at least during a part of the step b), a pH going from 1.5 to 3.0 or from the range 7.0 to 11.0, preferably 2.0-2.5 or 8.5-10.5.

4) The process of claim 1 in which the step of separation b) is done using a decanter, a disc centrifuge, a tubular centrifuge, a basket centrifuge or a rotary vacuum filter, preferably a decanter.

5) The process of claim 1 wherein the total mass content of starch hydrolysate and polysorbate added in step c), based on the total mass of dry matter of the soluble fraction, is between 0.5 and 10%, preferably between 2 and 4%.

6) The process of claim 1 wherein the starch hydrolysate:polysorbate ratio added in step c) in a mass ratio going from 0,66 to 15, by example, from 1 to 10, preferably from 2 to 8, more preferably from 2 to 5.

7) The process of claim 1 wherein the additive-containing soluble fraction is heated at a temperature going from 35 to 80° C., preferably from 60 to 70° C.

8) The process of claim 1 wherein the additive-containing soluble fraction is heated during from 30 to 150 minutes, advantageously from 30 to 120 minutes and preferably from 60 to 90 minutes.

9) The process of claim 1 wherein the using centrifuge, plate frame separate or membrane, to obtain a protein curd.

10) The process of claim 1 wherein the process comprises at least one step of washing the protein curd.

11) The process of claim 1 wherein the step of forming a protein precipitate is done by adjusting the pH of the heated additive-containing soluble fraction in the range going from 4.5 to 5.8, preferably from 5.0 to 5.5.

12) The process of claim 1 wherein it comprises a drying step consisting in a spray drying step.

13) A powder of an oat protein composition wherein said composition does not contain any traces of organic solvent, has protein content higher than 55%, has a extractable lipid content below 10% by weight on dry matter based on the total dry weight of the oat protein composition, a starch:soluble fiber mass ratio above 1 and has a mean particle size d-50 greater than 10 microns.

14) The powder of claim 13 wherein the composition contains more than 70% by weight of protein on dry matter based on the total dry weight of the oat protein composition, preferably more than 80% by weight.

15) The powder of claim 13 wherein the powder has parameters a* between −1 and 1, and L* above 80 when measured in the CIELAB color space.
